(19) United States
(12) Patent Application Publication
(10) Pub. No.: US 2011/0244554 A1
(43) Pub. Date: Oct. 6, 2011

(54) MECHANICAL DEVICE FOR DISPERSING BIOMASS POPULATIONS

(75) Inventor: ALBERT W. ALSOP, Wilmington, DE (US)

(73) Assignee: E. I. DU PONT DE NEMOURS AND COMPANY, Wilmington, DE (US)

(21) Appl. No.: 12/759,724

(22) Filed: Apr. 14, 2010

Related U.S. Application Data
(60) Provisional application No. 61/320,509, filed on Apr. 2, 2010.

(51) Int. Cl. C02F 3/34; C12M 1/00

(52) U.S. Cl. 435/262; 435/283.1

(57) ABSTRACT

A biomass dispersing system and method that breaks up biomass aggregates is disclosed. The system is designed to avoid cell damage and loss of cell viability while dispersing biomass aggregates for applications where biomass aggregates can cause operation problems. The dispersed biomass suspension obtained using the disclosed system will not plug the oil wells during biomass injection into underground wells.

### FIELD OF THE INVENTION

This disclosure relates to the field environmental microbiology. Specifically, it is related to a biomass dispersing device to produce finely dispersed substantially viable biomass suspensions. More specifically, this disclosure is of value for preparing substantially viable dispersed biomass for inoculating oil wells for microbial enhanced oil recovery and in bioremediation.

### BACKGROUND OF THE INVENTION

Microbial Enhanced Oil Recovery (MEOR) is a methodology for increasing oil recovery using microorganisms (Brown, L. R., Vadie, A. A., Stephen, O. J. SPE 59306, SPE/DOE Improved Oil Recovery Symposium, Oklahoma, 3-5 Apr., 2000). MEOR research and development is an ongoing effort directed to developing techniques to use microorganisms to modify crude oil properties to benefit oil recovery (Sunde, E., Beeder, J., Nilsen, R. K. Torsvik, T., SPE 24204, SPE/DOE 8^{th} Symposium on enhanced Oil Recovery, Tulsa, Okla., USA, Apr. 22-24, 1992).

One of the challenges in MEOR applications, following production of the required biomass and its storage, is to produce highly viable biomass populations for MEOR activity. Microbial biomass may be stored as a suspension for a short term either at room temperature or refrigerated or for longer periods in a frozen (WO2001/005941) or a lyophilized form (U.S. Pat. No. 3,261,761 and Rudge, R. H., Maintenance of Bacteria by freeze-drying, In: Maintenance of Microorganisms and Cultured Cells. A Manual of Laboratory Methods, 2^{nd} Edition, pp 31-44, ed: Kirsop, B. E. and Doyle, A., Academic Press, London). The biomass may be stored for either long term or short term in a concentrated (cell paste) or a dilute form suspended in various types of buffers with or without chemical additives. Stored biomass often goes through morphological and functional changes (Sharma, S., et al., Appl. Environ. Microbiol., 72: 2148-2154, 2006). Such biomass is usually more difficult to resuspend, partly due to chemical additives used to preserve its viability during storage (WO2001/005941) and partly due to changes that happen in the cells following storage, freezing and thawing. For example, cell mass may aggregate into gelatinous masses that resist resuspension and may plug fluid handling systems. Some cells may lyse and release DNA and form slimy and viscous masses (Sharma, S., supra).

Most often, stored biomass should be resuspended for further application and it is often imperative for the suspended biomass to be well dispersed. This is particularly important when biomass is intended for MEOR applications to avoid plugging the oil well following its injection. Further, the resuspended biomass should retain its original viability for an optimal MEOR process. The most common process used for biomass resuspension is gentle mixing or shaking of the biomass in a liquid medium. While gentle mixing preserves cell viability, it cannot produce a well dispersed biomass without many aggregates. Application of additional filtration to remove biomass aggregates, following gentle mixing, usually results in biomass loss and is costly to operate as frequent filter cleaning is required.

Preparation of dispersed biomass, however, requires mixing the biomass in the suspending fluid at high speed and shear which damages the biomass and results in cell lysis and loss of viability. For example, a high pressure homogenization technique commonly used for uniform dispersion of various particles in a liquid is too harsh on biomass and results in cell lysis. In fact this method is commonly used for cell disruption to release the cytoplasm's contents (Donsi, F., et al., Chem. Eng. Sci., 64: 520-532, 2009 and Anand, H., et al., Biochem. Eng. J., 35: 166-173, 2007).

Tangential flow (also known as cross flow) filtration utilizing membrane filters is often used to remove water and salt solutions from biomass suspensions. In these processes whole cells are retained while only submicron particles and solutions pass through the filter medium. Thus, this system allows collection of biomass but is not useful in breaking the biomass aggregates into finer particles containing individual cells. Application of various types of membranes to separate biomass aggregates often results in rapid membrane plugging or fouling by these aggregates (Aryal, R., et al., Sep. Pur. Technol., 67: 86-94, 2009).

U.S. Pat. No. 7,455,784 and U.S. Pat. No. 5,015,397 describe application of wedge wire and tangential flow in an apparatus used for separation solids from liquids from large volume of industrial fluids and during manufacturing processes.

WO2007028149 teaches removal of inert solids from a low-yield wastewater treatment process through combining wastewater with a biomass-containing sludge in a mainstream reactor to form a mixed liquor. A screening device, containing wedge wire filter elements, removes inert particles from 10 micrometers to 6,000 micrometers in size. The mixed liquor is then separated into a clear effluent and activated sludge.

The methods described above are useful in addressing solids in waste processes but do not address the production of finely dispersed substantially viable biomass suspensions for environmental applications.

The problem to be solved is to develop a device and a method to disperse aggregated biomass particles and to produce a finely dispersed biomass suspension while retaining the biomass viability. Lack of large aggregates in the finely dispersed substantially viable biomass suspension prevents plugging the well site following biomass injection into the well and thus does not disrupt the MEOR or bioremediation applications.

### SUMMARY OF THE INVENTION

This disclosure offers a solution to the problem stated above. Herein, a device and a method is disclosed which applies a biomass dispersing system comprising a biomass dispersing device to gently break biomass aggregates and to produce a finely dispersed viable biomass suspension.

In one embodiment, solutions containing the biomass containing aggregates are contacted with a biomass dispersing device comprising a filter element using tangential flow across the filter element. Larger aggregates in the feed flow either pass over the filter element unaffected or are abraded into smaller aggregates in the permeate flow. The retentate containing the larger particles flows back to the tank for reprocessing. Thus, valuable biomass contained within large aggregates in the retentate is retained and is circulated through the system for additional rounds of dispersion. All biomass is therefore retained until successfully dispersed. The permeate that contains finely dispersed viable individual cells and/or small biomass aggregates passes through the filter element. The relatively high tangential flow rate washes the filter element and prevents binding any biomass aggregates to it thus obviating the need for frequent cleaning of the filter element.

Accordingly the invention provides a method for delivering a viable biomass population to a target site comprising:
a) providing a biomass population suspended in a medium and contained in a biomass dispersing system;
b) flowing the biomass population of (a) through a biomass dispersing device whereby a finely dispersed viable biomass population is produced; and
c) applying the finely dispersed viable biomass population of (b) to a target site.

In another embodiment the invention provides a biomass dispersing system for producing finely dispersed viable biomass comprising:
a) a biomass population suspended in a medium and contained in a tank;
b) a biomass dispersing device; and c) an injecting means;
   wherein the biomass dispersing device communicates with the injecting means through a tubing means.
   In another embodiment the invention provides a method of preparing a substantially viable dispersion of a biomass population comprising: flowing a biomass population as a feed flow across a wedge wire device using tangential flow to produce a permeate flow comprising a substantially viable dispersion of a biomass population.
   In another embodiment the invention provides a method for enhancing oil recovery from an oil well site comprising:
a) providing a biomass dispersing system comprising a biomass population suspended in a medium;
b) flowing the biomass population of (a) through a biomass dispersing device whereby a finely dispersed viable biomass population is produced; and
c) injecting the finely dispersed substantially viable biomass population of (b) in to an oil well site; wherein the finely dispersed substantially viable biomass population is effective in releasing oil from the oil well site; and wherein
   1) the biomass dispersing system comprises a tank, pipelines, pumps, a biomass dispersing device and throttle valves; and wherein
   2) the biomass dispersing device comprises a wedge wire filter element contained in a casing; and wherein
   3) the biomass population comprises a microorganism selected from the group consisting of: Comamonas, Fusibacter, Marinobacterium, Petrotoga, Pseudomonas, Vibrio, Petrotoga, Thauera, Shewanella, Enterobactor, and Microbulbifer.

In another embodiment the invention provides a method for remediating an environmental site comprising:
a) providing a biomass dispersing system comprising a biomass population suspended in a medium;
b) flowing the biomass population of (a) through a biomass dispersing device whereby a finely dispersed substantially viable biomass population is produced; and
c) applying the finely dispersed substantially viable biomass population of (b) to an environmental site; wherein the finely dispersed substantially viable biomass population is effective in remediating the environmental site; and wherein
   1) the biomass dispersing system comprises a tank, pipelines, pumps, a biomass dispersing device and throttle valves; and wherein
   2) the biomass dispersing device comprises a wedge wire filter element contained in a casing; and wherein
   3) the biomass population comprises a microorganism selected from the group consisting of: Comamonas, Fusibacter, Marinobacterium, Petrotoga, Pseudomonas, Vibrio, Petrotoga, Thauera, Shewanella, Enterobactor, and Microbulbifer.

### BRIEF DESCRIPTION OF THE FIGURES

FIG. 1 depicts a vertical section of a typical oilfield injection well.

FIG. 2 shows a first view of the design and the fluid flow paths within a biomass dispersing device.

FIG. 3 shows a second view of the design and the fluid flow paths within a biomass dispersing device.

FIG. 4 shows a section of the wedge wire filter element.

FIG. 5 is a flow sheet illustrating various elements of a biomass dispersing system as follows.

FIG. 6 is a graph illustrating the particle size distribution of biomass immediately after thawing frozen blocks of biomass in the resuspension solution.

FIG. 7 is a graph illustrating the particle size distribution of the permeate following passage through the biomass dispersing device.

### DETAILED DESCRIPTION OF THE INVENTION

The present disclosure relates to a biomass dispersing system comprising a biomass dispersing device using tangential flow over a filter element to provide finely dispersed substantially viable biomass suspensions from stored aggregated biomass prior to application to a target site, as for example the inoculation of oil well sites for MEOR and bioremediation.

The following definitions may be used for the interpretation of the claims and specification:

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The terms “biomass” or “biomass population” are used interchangeably herein and refer to one or more populations of microorganisms. Typically such microorganisms are useful in the MEOR processes or in bioremediation. In some circumstances the biomass population comprises microorganism that form biofilms or biomass aggregates and require dispersion prior to inoculating a target site.

“Tangential flow” or “cross flow”, as used herein, refer to a flow that runs parallel to the surface of the filter element”.

The term “feed” or “feed flow” refers to a flow comprising medium, biomass and water that enters the biomass dispersing device through the feed flow inlet.

The term “retentate” as used herein, is feed that has passed over a filter element and refers to any biomass suspension, larger than the size of the filter element openings, that cannot not pass through it.

The term “permeate” or “permeate flow” as used herein, refers to the finely dispersed substantially viable biomass that has passed through the biomass dispersing device comprising single cells of biomass and fine biomass aggregates but are devoid of any large biomass aggregates.

The term “casing”, as used herein, means an outer layer surrounding a filter element.

A “filter element”, as used herein, refers to a surface containing openings which would allow passage of certain sizes of particles based on the size of the openings in the filter and prevents particles of larger size from passing through it.

The term “biomass dispersing device” refers to any device which disperses the biomass aggregates in a liquid to produce a finely dispersed biomass suspension without negatively impacting the integrity, mass and viability of the biomass.

“Finley dispersed substantially viable biomass suspension” or “finely dispersed substantially viable biomass population” are used interchangeably and refer to a biomass suspension in a liquid medium that contains either individual cells or fine cell aggregates less than 2 micrometers in size and is devoid of any large biomass aggregates that cannot pass through the filter element openings. In this context, viability is measured based on the number of colony forming units per milliliter (CFU/ml). The finely dispersed “substantially viable” biomass suspension retains greater than about 50% of its original CFU/m, where greater than about 60%-70% is typical and wherein greater than about 80% to 95% of its original CFU/m is preferred.

“Aggregated biomass” or “biomass aggregates”, as used herein, refer to a biomass that contains aggregated particles in sizes larger than the openings of the filter element.

“Target site” refers to any Environmental Site suitable for treatment by a biomass population. Target sites may be oil well sites, wherein the biomass is needed for microbially enhanced oil recovery (MEOR). Alternatively target sites may be any other above-ground or subterranean site where environmental bioremediation may be needed.

“Environmental site” refers to a target site that has been contaminated with persistent organic pollutants including hydrocarbons.

“Inoculating an oil well site” or “injecting into an oil well”, as used herein, involves injecting one or more finely dispersed substantially viable biomass populations into an oil well site or oil reservoir such that said biomass is delivered to the well or reservoir for their MEOR or bioremediation activity. “Oil well site”, as used herein, refers to a subterranean or sea-bed formation from which oil may be recovered.

A “bioremediation well site” is a location where a well has been drilled into the ground to access a subterranean location containing pollutants that are to be remediated or removed.

The present invention relates to the use of a biomass dispersing system comprising a biomass dispersing device for the production of substantially viable dispersed biomass populations prior to application of the biomass to a target site. The biomass populations of the present invention are typically suspensions of bacteria, fungi or yeast that are useful in applications for oil release at oil wells or bioremediation of environmental sites. The function of the biomass dispersion device is to disperse microbial cells that have formed or may form aggregates or biofilms prior to their application to the target site. Biomass suspensions that are not adequately dispersed may cause fouling or plugging of the dispersing system components or plugging of the subterranean formations of the target site.

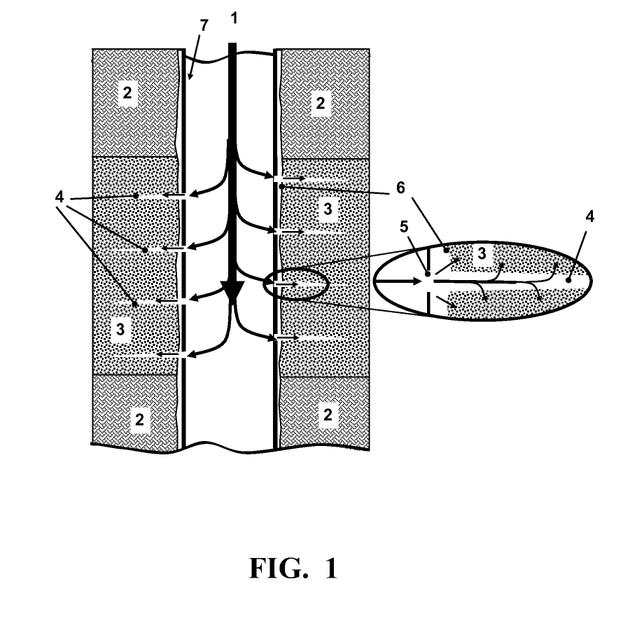

A typical oilfield injection well is shown in FIG. 1. The injected fluid into the well (1) is expected to flow from the surface injection point down the well into the desired location. The well casing or pipe (7) carries the injection fluid from the injection point to the desired location. The fluid is constrained by the non-permeable rock strata (2) that lies above and below the permeable rocks or matrix (3). Perforations and fractures in the permeable strata (4) facilitate the fluid flow into the permeable matrix. Perforations (6) that exist in the well casing allow the injection fluid to flow from within the well casing to the permeable matrix via the interface between the injection fluid and the permeable matrix. In this system, large aggregates of biomass can accumulate and plug the perforations (6) and thus prevent the fluid flow into the permeable matrix (3) and interfere with the oil recovery process—hence the need for an effective system to disperse aggregated biomass prior to application to an oil well site.

### The Biomass Dispersing System

The biomass dispersing system of the current disclosure comprises the biomass dispersing device and additional components such as valves, pumps, a tank and pipes. The biomass dispersing device is an element that has the ability to render a biomass population, typically comprising microbial cells, that are aggregated, may become aggregated or form biofilms, into a fine suspension of substantially viable cells that are suitable for application to a target site.

A multiplicity of methods may be use in the dispersing device to effect adequate biomass dispersion including filtration, ultrasonic energy and high shear mixing, examples of which are known in the art; for example, dispersing solids in liquid contained in a tank using a rotating agitator (Chemical Engineer's Handbook, Ed: Perry, 8th edition, section 18—Liquid-Solid Operations and Equipment, section 18-5); or using ultrasonic devices (U.S. Pat. No. 3,165,299); or using rotor-stator devices (U.S. Pat. No. 6,869,212) and using high pressure homogenization devices (U.S. Pat. No. 4,585,357). These devices rely on introduction of energy into a mixture of solids and a liquid to disperse the solids. Care needs to be taken in using these methods to preserve viability of the cells and a compromise between sufficient energy to disperse particles and too much energy that degrades or damages particles must be made.

Alternatively ultrasonic energy may be used for the dispersion. Ultrasonic energy (typically 20-50 kilo Hertz) may be applied to the particles suspended in a medium. However, this method, while very effective, employs high energy and biomass particles are particularly susceptible to the shearing effect produced during mixing, pumping and from ultrasonic energy. Consequently care must be taken to modulate the amount of energy used to maintain cell viability.

Another method of dispersion aggregated biomass employs filtration. Filtration is a mechanical or physical operation used for the separation of solids from fluids (liquids or gases) by passing the mixture of particles and the liquid through a porous medium of specific size. Thus only the fluid and particles smaller than the openings in the filter can pass through it. Oversize solids in the fluid are retained.

Various types of filters may be used in the disclosed method, including but not limited to, woven screens (American Standards and Testing and Materials (ASTM) E2016-06 Standard Specification for Industrial Woven Wire Cloth), perforated sheets (ASTM E674-09 and E454.6165-1) and wedge wire screens (U.S. Pat. No. 5,064,536). In one embodiment, the filter element may comprise a woven wire mesh screen. In another embodiment the filter element may comprise a perforated metal sheet.

Wedge wire screens or wedge wire filters are preferred herein. The term “wedge wire” refers to a wire that is formed with a trapezoidal cross section. The term “wedge wire filter element”, as used herein, refers to a filter element comprising multiple wedge wires arranged in parallel such that the widest part of the parallel wedge wire cross sections are adjacent and form narrow slit openings that fluids may pass through, flowing from the narrowest portion of the slit to the widest portion of the slit.

The shape of the openings (hereafter “slit openings” for the wedge wire filter elements) are constructed so that liquid medium containing particles enters the narrowest part of the slit and passes through the slit openings to the widest part. Thus particles that are close to the size of the narrowest part of the slit pass through the wedge wire slit openings without being retained in the slit and/or plugging it. The wedge wire filter element may be made of stainless steel, monel, polymer, plastic or some other material not subject to corrosion in the solution used to suspend the biomass. Specifically the wedge wire filter element may be made of 316 or 316L stainless steel. The wedge wire screens are preferred in the disclosed method since they provide the unique added advantage of a reduced tendency to plug and the possession of sharp edged slits that assist in breaking up larger aggregates of biomass into smaller aggregates.

In one embodiment, the size of the slit openings in the wedge wire filter element may be from about 5 micrometers to about 100 micrometers. In another embodiment the size of the slit openings in the wedge wire filter element may be from about 10 micrometers to about 50 micrometers. In yet another embodiment, the size of the slit openings in the wedge wire filter element may be from about 20 micrometers to about 40 micrometers.

In one embodiment, a wedge wire screen may be used to construct the cylindrical wedge wire filter element. In another embodiment a flat wedge wire filter element may be employed with an appropriate casing design that allows tangential flow over the wedge wire filter element on one side and collection of permeate on the other side of the wedge wire filter element. In another embodiment, to provide sufficient flow to handle larger flows, multiple devices may be employed in parallel. In another embodiment, the biomass dispersing device may optionally comprise additional filter elements. Any filter element with openings approximately equal to or slightly larger than the largest allowable size desired in the permeate may be used.

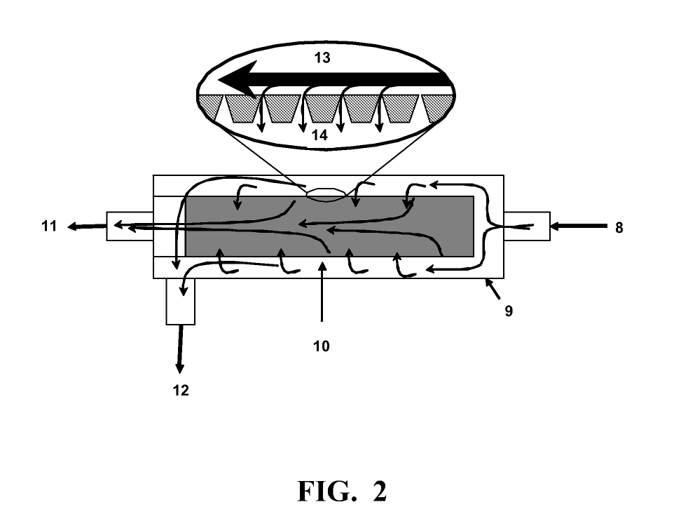

In one preferred embodiment the biomass dispersion device is a wedge wire filter as illustrated in FIG. 2, and comprises a casing (9) and a wedge wire filter element (10). Other additional elements of the biomass dispersing device comprise a feed flow inlet (8), an outlet for permeate (11), and an outlet to take the retentate (12) back to a tank holding the biomass suspension. An inset view shows the tangential flow (13) of the biomass suspension in an aqueous medium over the filter element with individual cells and finer aggregates penetrating the filter element and producing the permeate (14). The retentate (12), containing the suspension of the larger biomass aggregates, leaves the biomass dispersing device and circulates back to the tank. The flow of the feed and retentate over the outer surface of the filter element clean the filter element and prevent its plugging.

The casing (9) may be made of any material not subject to corrosion in the solution used to suspend the biomass and of sufficient strength to contain the fluid at the recirculation pump exit pressure. In one embodiment, the casing may be made of materials such as stainless steel, monel, plastic or polymer. In a preferred embodiment, stainless steel is used for the casing since it tolerates high pressure and corrosive environments. The casing of the biomass dispersing device disclosed herein may be constructed in a cylindrical shape to allow feed flow to enter the annular passage defined by the outside of the filter element and the inner wall of the casing.

The space between the casing (9) and the filter element (10) plays a significant role in determining the tangential velocity of the biomass suspension across the filter element. The ratio of the volumetric feed flow to the cross sectional area of the casing annulus determines the velocity of the feed or retentate stream across the filter element. In one embodiment, the space between the casing and the filter element may be less than about 26 millimeters and typically about 25.4 millimeters. Preferably the space between the casing and the filter element may be about 10 millimeters or less. In a preferred embodiment, the space between the casing and the filter element may be from about 2 to about 5 millimeters.

The geometry of the biomass dispersing device (FIG. 2) creates an annular volume (annulus) outside of the filter element (10) and inside the casing (9) that allows the feed flow to pass along the outside of the filter element and be collected at the end of the casing as retentate. Retentate is returned to the tank for recirculation through the biomass dispersing device. In this method, the permeate (14), containing finely dispersed biomass aggregates, flows through the slit openings of the wedge wire filter element to the permeate outlet (11).

A notable design factor for the operation of the biomass dispersing device disclosed herein is the tangential flow along the filter element which removes solids that may plug the filter element. Additionally the tangential flow cleans the outer surface of the filter element and prevents accumulation of biomass on the filter element surface.

The cross section of the annulus between filter element and the casing and the total liquid recirculation or feed rate determines the tangential velocity of the biomass suspension across the filter element. The tangential velocity provides for cleaning of the filter element and for shear and attrition of biomass particles that adhere to the wedge wire filter element slit openings. Normal velocity (i.e., the velocity into the wedge wire filter element through the slit openings) is determined by the size and number of slit openings in the wedge wire filter element and the total permeate flow rate. If the permeate flow rate is too high, relative to the tangential flow rate, filter plugging and inadequate particle attrition may result. Permeate flows first into the center of the wedge wire filter element where it collects and then moves to the permeate outlet due to the lower pressure at the permeate outlet for flow out of the biomass dispersion device.

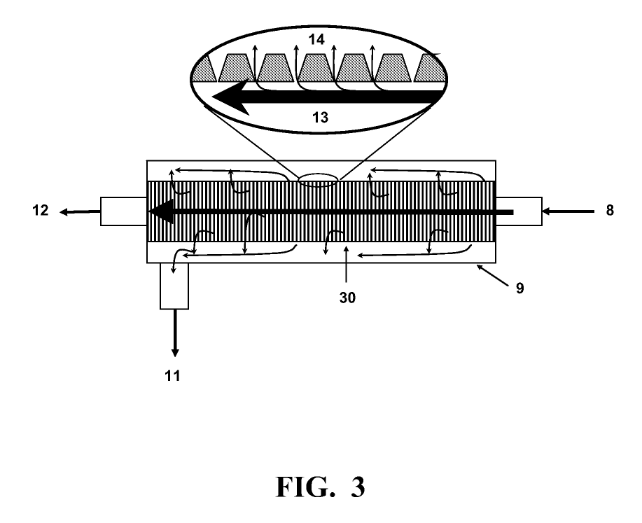

A variety of device geometries and flows may be used to accomplish the desired tangential flow to permeate flow ratio at the wedge wire filter element surface and for the collection of retentate and permeate. For example as shown in FIG. 3, feed flow inlet (8) may be directed down the center of the cylindrical wedge wire filter element (30) with the wedge wire filter element designed for the permeate (14) to pass from the center of the wedge wire filter element (30) to the outer annulus to be collected. Retentate (12) may be collected from the opposite inner end of the cylindrical wedge wire filter element from where the feed (8) enters and permeate (11) exits at the lower permeate outlet port.

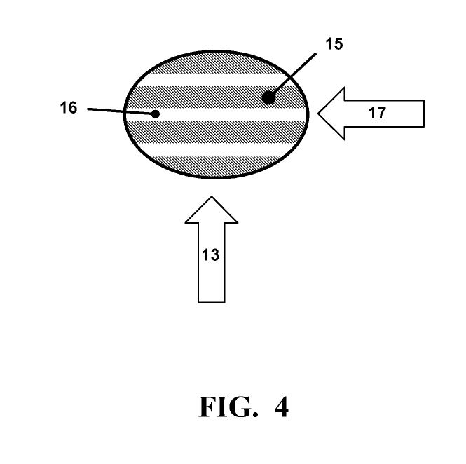

FIG. 4 depicts tangential flow that passes across (13) the wedge wire filter element slit openings (16). This geometry is preferred since it allows maximum attrition of biomass aggregates. If wedge wire filter element fouling or plugging occurs with a particular biomass or attrition is too severe with tangential flow across (13) the wedge wire slit openings (16) then a design to produce flow along (17) the wedge wire slit openings may provide better cleaning and a lower, less aggressive attrition of the biomass.

The wedge wire filter element design (FIG. 4), as used herein, has a high velocity tangential flow across (13) the slit openings in the wedge wire filter element (10) formed by the wedge wires (15). Such high velocity tangential flow maximizes the shear effect on any large biomass aggregates that adhere to the wedge wire filter element. To prevent plugging of the wedge wire filter element, the tangential velocity may be increased by either the geometry of the outer flow annulus and/or by increasing the volume of the feed passing through the device. The wedge wire filter element may be designed to allow the tangential flow to be along to the openings between wedge wires (17). This design improves the cleaning efficiency, but reduces biomass aggregate attrition.

To minimize plugging of the wedge wire filter element slit openings, it is desirable to limit the permeate flow rate to a small fraction of the tangential flow rate. For the current method values of permeate flow rate to feed flow of 1% to 90% of the feed flow rate may be used. Specifically values of permeate flow rate to feed flow rate of 1% to 50% of the feed flow rate may be used. More specifically values of permeate flow rate to feed flow rate of 1% to 5% of the feed flow rate may be used. If the permeate flow rate is allowed to get too large relative to the tangential flow rate then plugging of the wedge wire filter element slit openings may result. If permeate flow rate is too low relative to feed flow rate then excessive biomass attrition and possible loss of biomass viability may result.

### Other Components

Other suitable components for the current biomass dispersing system comprise: a tank, pumps, flow regulating or throttle valves, various pipes or hoses to transfer the biomass to the biomass dispersing device, and various hoses or pipes to transfer the larger aggregates of biomass in the retentate back to the tank to be recycled through the biomass dispersing device. Additional pipes or hoses convey the dispersed biomass permeate to a pump to facilitate its injection into an oil well site.

### The Tank

The tank which holds the biomass for dispersion, may also contain an aqueous solution comprising mineral salts, trace elements, and nutrients suitable for the maintenance and/or growth of the biomass. Depending on the environment of the tank and the nature of the biomass in the tank, heating or cooling capabilities may be required to establish and maintain a suitable temperature for the biomass. Material used for construction of the tank may be dictated by the nature of the environment required by the microorganisms. Stainless steel or polypropylene containers, commonly used for oil field applications, may be used. The “totes” or “LiquiTote” tanks (Hoover Material Handling Group Inc., Houston, Tex.), which are available in a variety of materials and sizes of 500 to 1,500 liter capacity, are well suited for application in this method. Truck mounted tanks, often used to transport chemical solutions, are also suitable tanks in this instance. Additive elements such as heater or cooler controls, level monitors, agitators and possibly aeration equipment may be added to the tank to sustain the biomass viability during processing. In one embodiment, a tank truck or a tank trailer may be used with the transfer pump mounted on the tank truck or the tank trailer.

### The Pumps

In the present biomass dispersing system, a centrifugal pump is used preferentially for pumping the biomass from the tank through the biomass dispersing device. The centrifugal pump provides additional shear to the biomass suspension during pumping and may increase the attrition rate and size reduction of the biomass aggregates. However, in cases where the biomass is sensitive to the high shear rate of a centrifugal pump and cell death results, a different type of pump with lower shear rate may be used. A wide variety of other pump types may be employed as needed with sufficient volumetric flow rate and discharge pressure and a suitable degree of liquid shear in the pump for the particular biomass being suspended. Examples of pumps that may be used in the disclosed biomass dispersing system include, but are not limited to, vane pumps, peristaltic pumps, gear pumps and diaphragm pumps.

In one embodiment, a positive displacement pump is used to transfer the permeate containing finely dispersed biomass to the injection site of the oil well. Additional pumps that may be useful for this purpose include, but are not limited to, triplex pumps, and multistage centrifugal pumps.

### The Valves

Flow regulating or throttle valves located at the pump's discharge may be replaced with a variable speed control device on the pump to regulate the pump speed and recirculation flow rate. In one embodiment, a throttle valve may be used to regulate the flow of permeate. In another embodiment a variable speed positive displacement pump may be used for this purpose.

### The Inlets and Outlets

The feed inlets and the permeate outlets provide for flow into and out of the biomass dispersing system. The inlets and outlets may be made of any commonly utilized mechanical piping or tubing connection compatible with the requirements of the environment that the system is used in. In one embodiment, the piping or hoses may be made of high pressure flexible hoses. In another embodiment, the piping or hoses may be made of rigid pipes with appropriate pressure rating.

### Biomass

The biomass useful for this application may comprise various classes of facultative aerobes, nitrifiers, obligate anaerobes, denitrifiers, and facultative anaerobes. The resuspended biomass may comprise only one particular species or may comprise two or more species of the same genera or a combination of different genera of microorganisms. Examples of various species of microorganisms useful in this application include, but not limited to: Comamonas, Fusibacter, Marinobacterium, Petrotoga, Pseudomonas, Vibrio, Petrotoga, Thauera, Shewanella, Microbulbifer, and Enterobacter.

The biomass may be produced under aerobic or anaerobic conditions depending on the particular microorganism(s) used. Techniques and various suitable growth media for growth and maintenance of aerobic and anaerobic cultures are well known in the art and have been described in “Manual of Industrial Microbiology and Biotechnology” (A. L. Demain and N. A. Solomon, ASM Press, Washington, D.C., 1986) and “Isolation of Biotechnological Organisms from Nature”, (Labeda, D. P. ed. p 117-140, McGraw-Hill Publishers, 1990).

The biomass used in this application may form biofilms on the surface of the biomass dispersing device. The term “biofilm” means a film or a coating, made of extracellular polymers formed by microorganisms, in which microorganisms are embedded and which adheres to surfaces. The biomass dispersing devise disclosed herein is designed such that any adhering or soft particles of biomass aggregates or biofilm that adhere to the filter screen are sheared off as described below.

In an embodiment, the microbial population used herein comprises Pseudomonas stutzeri (ATCC NO: PTA-8823) described in the commonly owned and copending application Ser. No. 12/105,769 and Shewanella putrefaciens (ATCC NO: PTA-8822) described in the commonly owned and copending application Ser. No. 12/105,690. Contents of both these applications are incorporated herein by reference in their entireties.

According to the present method, prior to inoculating an oil well site, the stored biomass is dispersed into a viable biomass population, using the disclosed biomass dispersing system, as described below.

### Operation of the Biomass Dispersing System

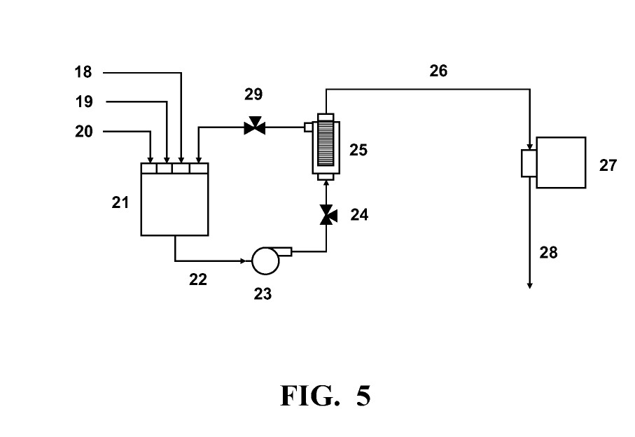

FIG. 5 depicts the geometry of various components of the biomass dispersing system disclosed herein comprising: Inlets (18, 19, and 20) deliver medium, biomass and water respectively to a tank (21) to produce a biomass suspension. A centrifugal pump (23) transfers the biomass suspension to the biomass dispersing device (25) via tubing or pipeline (22). A throttle valve (24) controls the flow of the biomass suspension to the biomass dispersing device. The finely dispersed viable biomass (permeate) is removed from the biomass dispersing device via pipelines (26 and 28), while the flow of the permeate is regulated by the positive displacement pump (27). Aggregates of biomass that do not pass through the wedge wire filter element are returned to the tank via a pipeline and a back pressure valve (29).

During operation of the biomass dispersing system, following addition of the mineral salts medium and water to the tank (21), the contents may be circulated through the biomass dispersing system several times if required to insure dissolution of the mineral salts medium. The temperature of the medium may be adjusted using an immersion heater, by ambient cooling or by addition of hot or cold liquids. Biomass may be added to the tank as frozen blocks, a concentrated suspension or as a dried solid and circulation is continued until the biomass has thawed and/or mixed with the medium. Following completion of thawing and mixing, the positive displacement pump (27) is started and the pump speed adjusted for the desired permeate flow. To increase aggregate dispersion, the biomass suspension may be allowed to circulate through the system using a slower permeate flow. Faster permeate flows may be used to facilitate delivery of the finely dispersed viable biomass to the permeate outlet (28). Aggregates of the viable biomass that do not pass through the openings of the wedge wire filter element slit openings are returned to the tank via a pipeline and a back pressure valve (29). “Tubing means” refers to any tubing or pipeline with suitable pressure rating and material of construction that can withstand the pressure employed during the operation of the current device and the chemical composition of the processed solutions. The permeate, containing the dispersed biomass population, is transferred via tubing or pipeline to the injecting means to be used in the oil well treatment or application for any other target site. The term “injecting means” as used herein, refers to any mechanical device like a centrifugal pump, a piston pump, gear pump or any other device, commonly known in the art, for pressurizing and transporting a fluid from a low pressure environment into a higher pressure environment, that is used to introduce the finely dispersed viable biomass into the oil well.

The design of the casing for the wedge wire filter element keeps the velocity of the fluid across the wedge wire filter element as high as possible subject to pressure drop limitations. This is done by minimizing the cross sectional area available for fluid flow of the annulus defined by the inner surface of the cylindrical casing and the outside surface of the cylindrical wedge wire filter element. A smaller cross sectional area for flow results in a higher liquid velocity for a given volumetric feed rate. This is done to maximize the cleaning effect of the high tangential velocity and to maximize attrition of biomass aggregates. The permeate, which has passed through the wedge wire filter element and contains the finely dispersed viable biomass, leaves the system via a pipeline (26 and 28) to be used as desired. The solution containing the biomass aggregates passes across the wedge wire filter element and is returned to the tank for further circulation.

### EXAMPLES

The present biomass dispersing system is further defined in the following Example. It should be understood that this Example, while indicating the preferred embodiments of the invention, is given by way of illustration only. From the above discussion and this Example, one skilled in the art can ascertain the essential characteristics of this invention, and without departing from the spirit and scope thereof, can make various changes and modifications of the invention to adapt it to various uses and conditions.

The meaning of abbreviations used in Example 1 is as follows: “min” means minute(s), “g” means gram(s); “ppm” means parts per million; “LPM” means liter per minute; “CFU/ml” means colony forming units per milliliter; “MPN” means Most Probable Number; “kg” means kilogram; “° C.” means degrees Celsius; “%” means percent; “SS” means stainless steel; “OD” means outer diameter; “g/L” means gram per liter; “wt %” means weight percent; “GPM” means gallon per minute; “μm” means micrometer(s) or micron(s); “wt %” means weight percent; “gal” means gallon(s).

### Example 1

### Dispersion of Biomass Using the Biomass Dispersing System

A biomass dispersing device (FIG. 2) was constructed using a stainless steel casing which housed an approximately 1.32 inch OD×8 inch long (3.35×20.32 cm) wedge wire filter element in it. The wedge wire filter element was constructed by the manufacturer by wrapping the stainless steel wedge wire around the support structure to establish 002″+0.002 inch/−0.001 inch ((0.0508+0.00508/−0.00254 cm) slit openings in the wedge wire filter element with a 1.07 square inch (6.903 cm) open area (3.23% open area for permeate flow). The wedge wire was then spot welded to the inner support to maintain a consistent slit opening dimension. As shown in FIG. 2 the wedge wire filter element was constructed with the narrow opening of the slit opening on the outside of the wedge wire filter element to allow the permeated liquid flow from the outside of the wedge wire filter element to the inside. This geometry is commonly practiced to avoid particles from becoming wedged in the slit openings thus plugging the filter element.

Frozen biomass was prepared using Pseudomonas stutzeri (ATCC NO: PTA-8823) as described in the commonly owned and copending application Ser. No. 12/105,769 and Shewanella putrefaciens (ATCC NO: PTA-8822) as described in the commonly owned and copending application Ser. No. 12/105,690 both of which are herein incorporated by reference on their entirety. Upon completion of fermentation, the biomass was concentrated by microfiltration of the broth to approximately 50 g/L of dried cell weight and glycerol (approximately 10 wt %) was added to the concentrated biomass. The broth was transferred to sterile bags containing medium, frozen by packing them in dry ice and stored at −80° C. until needed.

The schematics of the biomass dispersing system is shown in FIG. 5. Inlets (FIGS. 5, 1, 2 and 3) were used for manual addition of the medium components, the frozen biomass blocks (approximately 600 g) and water to the tank (4). The 35 gal (132 liters) polypropylene flat bottom tank was connected to an ˜7 GPM centrifugal pump (FIG. 5, 6) via a ½ inch (1.27 cm) stainless steel line (FIG. 5, 5). The pump was connected via a ½″ stainless steel line to a throttle valve (FIG. 5, 7) (Swagelok SS-A3ES8, 29500 Solon Road, Solon, Ohio) then through the same line to the inlet of the dispersing device (FIG. 5, 8). A ½″ inch stainless steel line connected to a throttle valve (FIG. 5, 12) was used to return the retentate flow from the dispersing device to the tank for recirculation. Flexible tubing (FIG. 5, 9), connected to the permeate port of the biomass dispersing device, allowed passage of the permeate from the device to a positive displacement pump (FIG. 5, 10) (Welch model 3200 peristaltic pump, Welch Rietschle Thomas, 7301 North Central Avenue, Skokie, Ill.). The permeated flow rate was regulated by the positive displacement pump speed. The permeate was collected via the plastic tubing (FIG. 5, 11).

To perform the experiment, the tank was filled with 13.5 gal (51 liters) of the dilute mineral salt solution to sustain the biomass (see Table 1) during processing. The contents of the tank were recycled between the tank and the biomass dispersing device using the centrifugal pump (FIG. 5, 6) and valves (FIG. 5, 7) and (FIG. 5, 12) to allow mixing of the biomass and the mineral salts solution upon thawing of the frozen biomass.

TABLE 1
Mineral salt solution used for biomass dispersion

| Ingredients | ppm |
|---|---|
| NaNO₃ | |
| Lactic Acid | 201.0 |
| NaLactate (NaC₃H₅O₃) | 250.0 |
| Sodium Bicarbonate | 1377.0 |
| NaCl | 11560 |
| CaCl*6H₂O | 394 |
| MgCl₂*6H₂O | 217 |
| Sodium Acetate | 500 |
| Oil | 50 |
| Sodium Bicarbonate | 1377 |
| CaCl₂*\hat{ }H₂O | 394 |
| MgCl₂*6H₂O | 217 |
| BaCl₂*2H₂O | 32 |
| KCl | 90 |
| SrCl₂*6H₂O | 15 |
| LiCl | 6 |
| NaCl | 11560 |

An electrical immersion heater was placed in the tank during recirculating the mineral salt solution to raise the water temperature to 27° C. The pH of the circulating water was maintained at 7.5±0.2 using either sodium hydroxide or nitric acid solution as required. Five blocks of frozen biomass with a total weight of 2.9 kg (approximately 60 g dried cell wt/L) were added to the tank. The mineral salt solution was recirculated through the biomass dispersing system at approximately 6.0 GPM (27.2 liter per min) for approximately 36 min to thaw the frozen blocks in the tank. As the frozen biomass blocks thawed, the biomass suspension in the mineral salts solution started to form. When biomass thawing was completed, the permeated cell suspension which had passed through the slit openings of the wedge wire filter element was removed at a volumetric flow of approx. 1.9 LPM. At this stage, 100 ml samples were withdrawn from both the tank and the permeate and the number of viable cells (CFL/ml) in them were determined. Later, the permeate flow was increased to 19 LPM and samples were removed for CFL/ml count again. When the level of biomass suspension in the tank had dropped too low to continue pumping, the centrifugal and the peristaltic pumps were stopped and the biomass dispersing device was inspected for evidence of plugging.

During the circulation of the tank's contents and filtration process no indications of increasing pressure drop through the wedge wire filter element was observed. Examination of the wedge wire filter element and the tank at the end of the experiment showed no visible accumulation of biomass in the tank or on the wedge wire filter element. Results of analysis for viable cell counts of the suspension in the tank and the permeate samples are shown in Table 2.

TABLE 2
CFU/ml in samples from the tank and the permeate

| | Tank | Permeate |
|---|---|---|
| 95% CI = ±0.57 | Log10 MPN | Log10 MPN |
| Permeate flow | Log10 | Log10 |
| rate and time | (CFU/mL) | (CFU/mL) |
| microbes in the | 2.8 | |
| tank prior to | | |
| biomass | | |
| addition | | |
| 1.9 LPM t = 1 min | 8.8 | 8.8 |
| 1.9 LPM t = 10 min | 9.1 | 9.4 |
| 19 LPM t = 2 min | 8.8 | 9.1 |

CI = confidence interval;
MPN = Most Probable Number in CFU/ml;
t = time,
CFU = Colony Forming Units

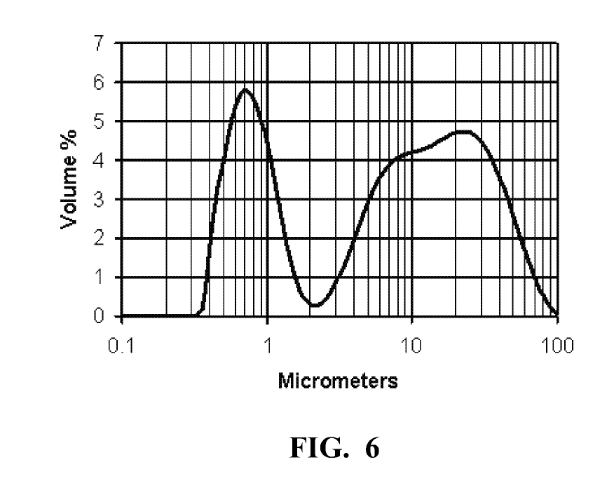

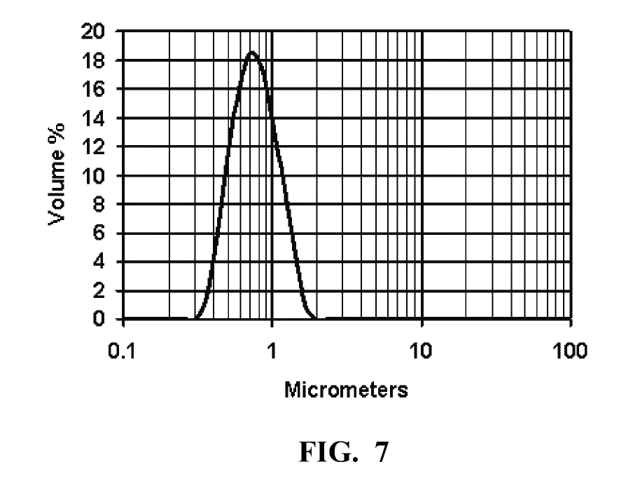

As shown in Table 2 no statistically measureable differences were observed in the CFU/ml of the biomass suspension in the tank and the permeate. Underlining that the cells in the permeate had retained their original viability. Measurement of particle sizes in the biomass suspensions using a Malvern Mastersizer™ 2000 (Malvern Instruments Inc., Westborough, Mass.) were performed using two samples. One sample was taken immediately after thawing frozen blocks of biomass in the resuspension solution (FIG. 6) and the other sample was of the permeate (FIG. 7) from the biomass dispersing device. Significant differences between these two biomass suspensions were observed. In the thawed frozen block biomass, prior to dispersion, large volumes of undispersed biomass existed as aggregates greater than 2 microns in size. On the other hand, particle sizes in the permeate sample were consistent with suspension of individual microorganisms with no significant aggregates of greater than 2 μM in size (FIG. 7 and Table 3).

TABLE 3
Characteristic particle sizes following passage through the biomass
dispersion device

| | Permeate |
|---|---|
| | Particle |
| | size (μm) |
| d10 | 0.530 |
| d50 | 0.816 |
| d90 | 1.283 |

D10 - Diameter of particle (μm) where 10 wt % of particles are smaller and 90 wt % of particles are larger.
D50 - Median of the particle size distribution, diameter of a particle where 50 wt % of particles in a sample are smaller and 50 wt % are larger.
D90 - Diameter of particle where 90 wt % of particles are smaller and 10 wt % of particles are larger. Thus the data presented herein confirms that the dispersed biomass population produced using the biomass dispersing device disclosed herein contained particles below 2 micrometers in size and were devoid of any aggregates.

What is claimed is:

1. A method for delivering a substantially viable biomass population to a target site comprising:
a) providing a biomass population suspended in a medium and contained in a biomass dispersing system;
b) flowing the biomass population of (a) through a biomass dispersing device whereby a finely dispersed substantially viable biomass population is produced; and
c) applying the finely dispersed substantially viable biomass population of (b) to a target site.

2. The method of claim 1 wherein the target site is selected from the group consisting of an oil well site, a bioremediation well site and an environmental site.

3. The method of claim 1 wherein the biomass dispersing system comprises a tank, pipelines, pumps, a biomass dispersing device and throttle valves.

4. The method of claim 1 wherein the biomass dispersing device of (b) disperses the biomass population by a means selected from the group consisting of: filtration, ultrasonic energy and high shear mixing.

5. The method of claim 4 wherein the biomass dispersing device comprises a filter element contained in a casing.

6. The method of claim 5 wherein a gap of less than about 26 millimeters exists between the casing and the filter element.

7. The method of claim 5 wherein the filter element is selected from the group consisting of: a woven screen element, a perforated sheet element and a wedge wire filter element.

8. The method of claim 7 wherein the wedge wire filter element comprises slit openings of about 5 micrometers to about 100 micrometers.

9. The method of claim 7 wherein:
a) the filter element is a wedge wire filter element, and
b) the biomass population suspended in the medium is passed across the wedge wire filter element as a feed flow using tangential flow, and exits the filter element as a permeate flow.

10. The method of claim 9 wherein the rate of flow of the permeate flow ranges from about 1% to about 90% of the rate of flow of the feed flow.

11. The method of claim 1 wherein the biomass population comprises microorganisms which form biofilms or biomass aggregates.

12. The method of claim 11 wherein the biomass population comprises a microorganism selected from the group consisting of: Comamonas, Fusibacter, Marinobacterium, Petrotoga, Pseudomonas, Vibrio, Petrotoga, Thauera, Shewanella, Enterobactor, and Microbulbifer.

13. The method of claim 12 wherein the microorganism is selected from the group consisting of: Pseudomonas stutzeri and Shewanella putrefaciens.

14. A biomass dispersing system for producing finely dispersed substantially viable biomass comprising:
a) a biomass population suspended in a medium and contained in a tank;
b) a biomass dispersing device; and
c) an injecting means;
wherein the biomass dispersing device communicates with the injecting means through a tubing means.

15. The biomass dispersing system of claim 14 wherein the biomass dispersing device comprises a filter element contained in a casing.

16. The biomass dispersing system of claim 15 wherein the filter is a wedge wire filter element.

17. The biomass dispersing system of claim 14 wherein the biomass population comprises a microorganism selected from the group consisting of: Comamonas, Fusibacter, Marinobacterium, Petrotoga, Pseudomonas, Vibrio, Thauera, Petrotoga, Shewanella, Enterobactor, and Microbulbifer.

18. A method of preparing a substantially viable dispersion of a biomass population comprising: flowing a biomass population as a feed flow across a wedge wire device using tangential flow to produce a permeate flow comprising a substantially viable dispersion of a biomass population.

19. The method of claim 18, wherein the rate of flow of the permeate flow ranges from about 1% to about 90% of the rate of flow of the feed flow.

20. The method of claim 1, wherein the wedge wire comprises slit openings of from about 5 micrometers to about 100 micrometers.

21. The method of claim 18, wherein the biomass population is selected from the group consisting of Comamonas, Fusibacter, Marinobacterium, Petrotoga, Pseudomonas, Vibrio, Thauera, Petrotoga, Thauera, Shewanella, and Microbulbifer.

22. A method for enhancing oil recovery from an oil well site comprising:
a) providing a biomass dispersing system comprising a biomass population suspended in a medium;
b) flowing the biomass population of (a) through a biomass dispersing device whereby a finely dispersed viable biomass population is produced; and
c) injecting the finely dispersed substantially viable biomass population of (b) in to an oil well site; wherein the finely dispersed substantially viable biomass population is effective in releasing oil from the oil well site; and wherein
1) the biomass dispersing system comprises a tank, pipelines, pumps, a biomass dispersing device and throttle valves; and wherein
2) the biomass dispersing device comprises a wedge wire filter element contained in a casing; and wherein
3) the biomass population comprises a microorganism selected from the group consisting of: Comamonas, Fusibacter, Marinobacterium, Petrotoga, Pseudomonas, Vibrio, Petrotoga, Thauera, Shewanella, Enterobactor, and Microbulbifer.

23. A method for remediating an environmental site comprising:
a) providing a biomass dispersing system comprising a biomass population suspended in a medium;
b) flowing the biomass population of (a) through a biomass dispersing device whereby a finely dispersed substantially viable biomass population is produced; and
c) applying the finely dispersed substantially viable biomass population of (b) to an environmental site; wherein the finely dispersed substantially viable biomass population is effective in remediating the environmental site; and wherein
1) the biomass dispersing system comprises a tank, pipelines, pumps, a biomass dispersing device and throttle valves; and wherein
2) the biomass dispersing device comprises a wedge wire filter element contained in a casing; and wherein
3) the biomass population comprises a microorganism selected from the group consisting of: Comamonas, Fusibacter, Marinobacterium, Petrotoga, Pseudomonas, Vibrio, Petrotoga, Thauera, Shewanella, Enterobactor, and Microbulbifer.
